(19) United States
(12) Patent Application Publication
(10) Pub. No.: US 2004/0147881 A1
(43) Pub. Date: Jul. 29, 2004

(54) Huber needle with a manually movable sleeve member for containing its injection point

(76) Inventor: Dongchul D. Hyun, Brea, CA (US)

Correspondence Address: Kit M. Stetina, Esq., STETINA BRUNDA GARRED & BRUCKER, Suite 250, 75 Enterprise, Aliso Viejo, CA, 92656, US

(21) Appl. No.: 10/351,120

(22) Filed: Jan. 24, 2003

(51) Int. Cl. A61M 5/00

(52) U.S. Cl. 604/263; 604/110

(57) ABSTRACT

A safety needle assembly comprising a needle with a distal needle end having a sleeve member which has a distal sleeve end forming an aperture. The sleeve member is mounted to the needle by accommodating the same through its sleeve end's aperture. The sleeve member is axially movable toward the distal needle end until the needle becomes displaced from the aperture and the distal needle end is contained within the distal sleeve end.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

The present invention relates generally to medical needle assemblies, and more particularly to an improved needle assembly featuring a sleeve which is adapted to move along a length of a needle and contain its injection point therewithin so as to safeguard its user against a needle-stick injury during a needle withdrawal procedure.

Needle-stick injuries are common phenomena in needle application procedures. They are a significant problem that threatens the safety of many health-care workers. In particular, a great percentage of these injuries occur during withdrawals of the needles from patients.

Inadvertent needle sticking of the body, particularly the hand, is not only painful, but presents a very high risk for pathogen transmission as well. More specifically, a Huber needle is primarily utilized for venous access. In this regard, it is a likely medium for transferring various blood-borne pathogens such as hepatitis B, hepatitis C and HIV from the patient to the health-care worker.

In order to safeguard against such dangers of needle-stick injuries, various measures have been proposed in the health-care industry. One commonly known safety measure is the use of home-made needle guards which are essentially thickened coverings for the health-care worker's free hand. However, although these home-made needle guards may achieve their primary objective of preventing a needle-stick injury, they possess certain deficiencies which detract from their overall utility.

Perhaps the greatest deficiency of such home-made needle guards is the extreme inconvenience associated with their use. Because the home-made needle guards are merely thickened coverings which are typically designed at home for the sole purpose of protecting the free hand, they often lack the engineering sophistication that would facilitate the process of administering the Huber needle. Rather, these home-made needle guards usually interfere with such administration as they typically handicap the use of the health-care worker's free hand which is vital for securing the implanted IV port.

Thus, there exists a strong need in the health-care industry for a safety needle guard which can protect health-care workers against the dangers of needle-stick injuries in a convenient and user-friendly manner. More specifically, there exists a need for a safety needle guard which can achieve such objective without handicapping the use of the health-care worker's free hand to thereby facilitate the overall process of administering the Huber needle.

### BRIEF SUMMARY OF THE INVENTION

The present invention specifically addresses and alleviates the above-described deficiencies associated with the use of the prior art Huber needle guards. More particularly, the present invention is an improved needle assembly featuring a sleeve which is adapted to move along a length of a needle and contain its injection point therewithin. Such containment protects a user (i.e., health-care worker) from being unintentionally stuck by the needle's injection point, thus safeguarding the user against the dangers associated therewith. Although this sleeve is intended primarily for Huber needle applications, its use with other types of conventional needles (e.g., standard hypodermic syringes) is specifically contemplated herein.

In accordance with a preferred embodiment of the present invention, there is provided a safety needle assembly for safeguarding its user against the dangers of a needle-stick injury which are often presented during a needle withdrawal procedure. The safety needle assembly of the present invention features a needle intended for various applications such as delivering fluids and medications, drawing blood for diagnostic testing and/or infusing blood products. The needle is preferably a metallic Huber needle configured to access an implanted port and conduct any one of the applications just described above.

The Huber needle includes proximal and distal needle portions which are interconnected to each other by an intermediate needle portion thereof. The intermediate needle portion defines a sharp downward curvature which causes the proximal and distal needle portions to be disposed generally perpendicular to each other. The proximal needle portion is ultimately placed in communication with a conventional syringe or infusion pump via a flexible infusion tubing. The distal needle portion, on the other hand, defines a sharp injection point which is used for penetrating through a patient's skin and accessing the subcutaneous IV port implanted thereunderneath.

In the preferred embodiment of the present invention, a sleeve is engaged around the Huber needle. More specifically, the sleeve is engaged in rather loosely so that it is allowed to axially move (preferably manually) along the length of the needle. With the exception of the sleeve's proximal and distal ends, the body of the sleeve is preferably flexible enough to accommodate and axially move over the sharp curvature of the intermediate needle portion when traveling therealong.

Defined on one particular end of the sleeve body is a proximal sleeve end. Essentially, the proximal sleeve end's range of movement is substantially limited between the proximal and intermediate needle portions while the remainder of the sleeve body is permitted to axially travel down to the distal needle portion. In the preferred embodiment, the proximal sleeve end comprises a collar which is attached to the sleeve body. The collar is formed having sufficient rigidity to be used to axially push the sleeve along the length of the needle. However, its rigidity prevents axial travel beyond the sharp downward curvature transition of the intermediate needle portion. Hence, the collar is forced to stop once it reaches the intermediate needle portion from the proximal needle portion thereby preventing any further axial movement of sleeve downwardly along the distal needle portion.

A distal sleeve end is defined on the opposite end of the sleeve body. Unlike the proximal sleeve end, the distal sleeve end is free to move along the entirety of the needle including its intermediate and distal needle portions. In the preferred embodiment, the distal sleeve end comprises a cap which is fabricated from a rigid material and attached to the sleeve body. The cap includes an inner cap surface which is divisible into two surface halves (i.e., first and second surface portions).

An aperture is formed through the inner cap surface, and more particularly with respect to the second surface portion. However, the formation of the aperture through one particular surface portion is by design choice only as such aperture may be formed through the other surface portion or even between the two surface portions. In the preferred embodiment, an aperture of smaller size than the second surface portion is formed through a selected location of the second surface portion. Alternatively, the second surface portion comprises a void which constitutes to be the aperture of the second preferred embodiment. In the latter embodiment, a ridge is preferably elevated between the first and second surface portions.

The aperture serves to provide an opening which allows the distal needle portion of the Huber needle to be movably accommodated therethrough. As such, the sleeve can axially move along the length of the needle towards its distal needle end until the proximal sleeve end approaches the intermediate needle portion. When the sleeve stops its axial motion due to the proximal sleeve end encountering the bend of the intermediate needle portion, the distal needle end becomes displaced from or moves out of the aperture.

Upon such displacement from the aperture, the distal needle end portion of the Huber needle is contained within the distal sleeve end, that is, the cap of the sleeve. Although the distal needle end may be contained within the cap in any manner or fashion, it is preferably shifted away from the aperture and abuts the opposite surface portion from where the aperture is located. A slight compression force which exists between the Huber needle and the aperture operates to cause such shift.

In this position, the sleeve is prevented from moving back up the distal needle by abutment with the opposite surface portion. In the instance of the second embodied aperture, the presence of the elevated ridge prevents the distal needle end from moving back into the aperture.

In accordance with a preferred embodiment of the present invention, an assembly holder device may be optionally used with the safety needle assembly of the present invention. As will be explained, the holder device is utilized for securing the safety needle assembly to the patient's designated skin area. In particular, the assembly holder device features an expandible/compressible holder mechanism which is adapted to open up and expand in order to accommodate a portion of the sleeve therewithin.

Thereafter, the holder mechanism may close to a sufficient degree to compress the sleeve so as to securely engage the same. Such closing of the holder mechanism may be achieved in any manner but preferably is accomplished through folding of two generally rectangular platforms or wings which individually extend outward from the holder mechanism. The platforms may be taped to the patient's designated skin area so as to firmly mount the present safety needle assembly thereupon.

In operation, the distal needle end of the Huber needle is first inserted into the patient's designated skin area to access the IV port implanted thereunderneath. Optionally, the present needle assembly may be secured in place by engaging the assembly holder device thereto and taping its outwardly extending platforms about the patient's designated skin area.

After treatment via infusion into the IV port, the distal needle end of the Huber needle is then withdrawn from the patient. During this withdrawal procedure, the sleeve is pushed axially toward the distal needle end until the distal needle end portion becomes displaced from or out of the distal sleeve end's aperture. Such axial pushing is performed manually preferably with the non-free hand while the free hand is used to secure the implanted port. By manually moving the sleeve in this fashion, the distal needle end of the Huber needle is contained within the distal sleeve end, thus preventing any risk for a needle-stick injury to its user.

### BRIEF DESCRIPTION OF THE DRAWINGS

These as well as other features of the present invention will become more apparent upon reference to the drawings wherein:

FIG. 1 is a side view of a safety needle assembly constructed in accordance with a preferred embodiment of the present invention illustrating its sleeve member which is engaged around a Huber needle;

FIG. 2 is a side view of the safety needle assembly of FIG. 1 illustrating its distal sleeve end which contains a distal needle end therewithin subsequent to the sleeve member's downward transition along the Huber needle;

FIG. 3 is a top cross-sectional view of the distal sleeve end of FIG. 2 illustrating its aperture which is similarly sized as an adjacent first surface portion and separated therefrom via a ridge elevated therebetween;

FIG. 3A is a side cross-sectional view of the distal sleeve end of FIG. 3 illustrating the manner in which the distal needle end is contained therewithin subsequent to its displacement from the aperture;

FIG. 3B is a side cross-sectional view of the distal sleeve end of FIG. 3 illustrating the manner in which the distal needle end is accommodated through the aperture;

FIG. 4 is a side cross-sectional view of a distal sleeve end constructed in accordance with an alternate preferred embodiment of the present invention illustrating its aperture which is formed through one selected point of a second surface portion;

FIG. 4A is a side cross-sectional view of the distal sleeve end of FIG. 4 illustrating the manner in which a distal needle end is contained therewithin subsequent to its displacement from the aperture;

FIG. 4B is a side cross-sectional view of the distal sleeve end of FIG. 4 illustrating the manner in which a distal needle end is accommodated through the aperture; and

FIG. 5 is a perspective view of an assembly holder device featuring a holder mechanism for engaging the sleeve member of FIG. 1 and illustrating a pair of outwardly extending platforms or wings which are utilized for securing to a patient.

### DETAILED DESCRIPTION OF THE INVENTION

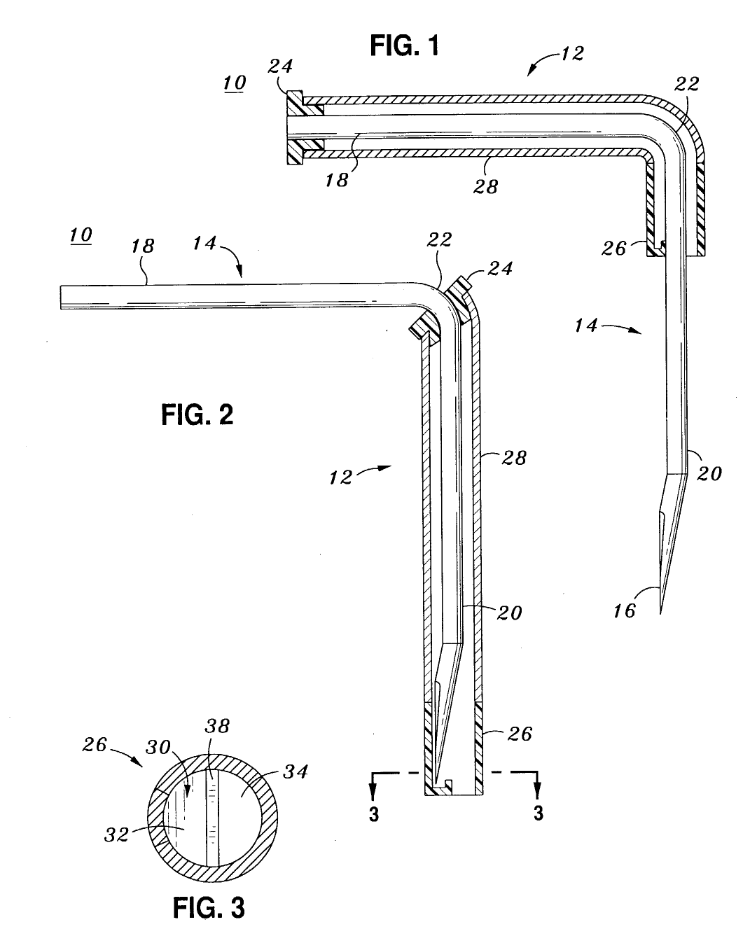

Referring now to the drawings wherein the showings are for purposes of illustrating preferred embodiments of the present invention only, and not for purposes of limiting the same, FIG. 1 illustrates a safety needle assembly 10 constructed in accordance with a preferred embodiment of the present invention. As indicated above, the present safety needle assembly 10 features a sleeve member 12 which moves axially along a length of a Huber needle 14 to securely contain its distal needle end 16 therewithin.

The sleeve member's ability to contain the distal needle end 16 internally yields a clear advantage to protect a user (i.e., health-care worker) against the dangers of a needle-stick injury which are often presented during a needle withdrawal procedure. Although the sleeve member 12 is intended primarily for Huber needle applications, its use with other types of conventional needles (e.g., standard hypodermic syringes) may be foreseeable.

Referring more particularly to FIGS. 1 and 2, the safety needle assembly 10 includes a metallic Huber needle 14 preferably fabricated from stainless steel. This needle 14 is primarily intended to carry out various tasks such as delivering fluids and medications, drawing blood for diagnostic testing, infusing blood products and the like. However, it should be noted herein that other types of tasks which involve accessing of the implanted port are specifically contemplated herein.

The Huber needle 14 comprises a proximal needle portion 18 and a distal needle portion 20 which are interconnected to each other by an intermediate needle portion 22 thereof. The intermediate needle portion 22 includes a sharp downward curvature or bend which causes the proximal and distal needle portions 18, 20 to be disposed in a generally perpendicular orientation. As is well known, the proximal needle portion 18 is ultimately placed in communication with an infusion pump via a flexible infusion tubing, for example (not shown). Furthermore, the distal needle portion 20 includes a sharply pointed distal needle end 16 which is used for penetrating through a patient's skin and accessing the IV port implanted thereunderneath (not shown). Preferably, the distal needle end 16 is a non-coring needle end.

A sleeve member 12 is positioned upon the Huber needle 14. The sleeve member 12 extends rather loosely around the needle 14 so that it is allowed to axially move along the length of the needle 14. With the exception of the sleeve member's proximal and distal ends 24, 26 which will be mentioned shortly, the body 28 of the sleeve member 12 is preferably constructed from a flexible material so that it may bend around the sharp curvature of the intermediate needle portion 22 during such axial movement.

Disposed on one end of the sleeve body 28 is a proximal sleeve end 24. It should be recognized that the proximal sleeve end's range of axial movement is substantially limited between the proximal and intermediate needle portions 18, 22. However, the rest of the sleeve body 28 is not so limited and is free to travel down to the distal needle portion 20.

Preferably, the proximal sleeve end 24 comprise a collar which is attached to the sleeve body 28. This collar is formed having sufficient rigidity to be used to manually push the sleeve member 12 axially along the length of the Huber needle 14. However, its rigidity is sufficient not to yield to (i.e., extend over) the sharp downward curvature of the intermediate needle portion 22. Hence, such collar is forced to stop upon reaching the intermediate needle portion 22 from the proximal needle portion 18 as its rigid construction prevents the collar to be pushed beyond that portion 22 (best shown in FIG. 2). In this respect, the length of the sleeve body 28 is sufficient to substantially extend from the intersection needle portion 22 down to the distal needle portion 20.

Referring now to FIGS. 2-4B, a distal sleeve end 26 is defined on the opposite end of the sleeve body 28. Unlike the proximal sleeve end 24, the distal sleeve end 26 is free to move along the entirety of the Huber needle 14 including its intermediate needle portion 22. In the preferred embodiment, the distal sleeve end 26 includes a cap which is attached to the sleeve body 28. The cap may be fabricated from any rigid material such as plastic or metal. This cap includes an inner cap surface 30. For the purposes of facilitating its description, the inner cap surface 30 is divided into two surface halves which will be referred to as first and second surface portions 32, 34.

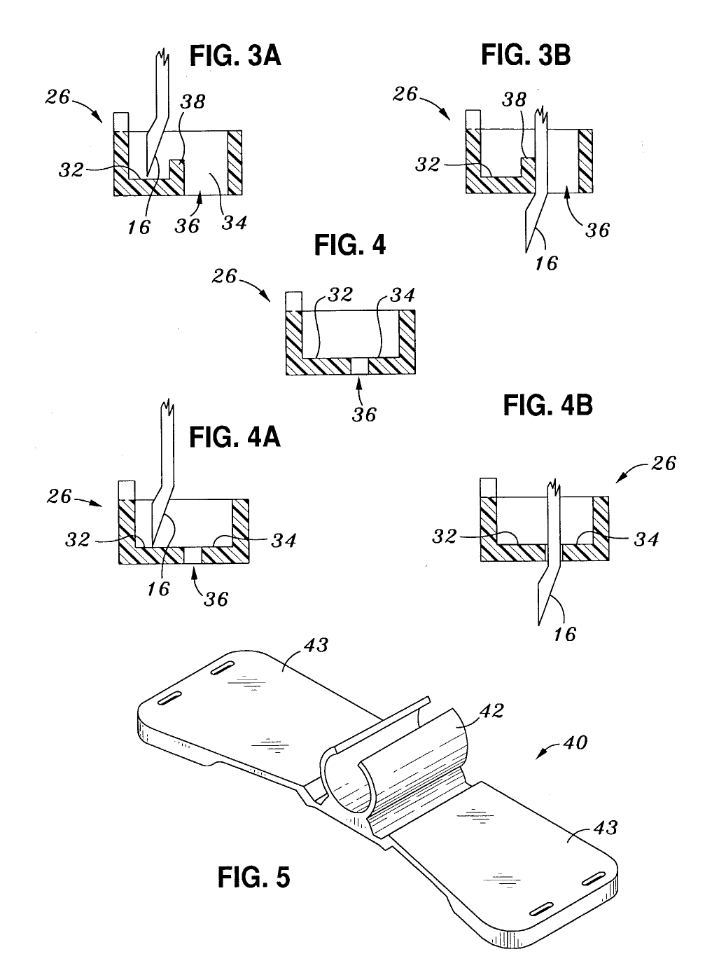

An aperture 36 is formed through the inner cap surface 30, and more particularly with respect to the second surface portion 34. However, the formation of such aperture 36 through one particular surface portion 34 is by design choice only, as such aperture 36 may be formed through the other surface portion 32 or even between the two surface portions 32, 34. In one preferred embodiment, an aperture 36 having a diameter smaller than the second surface portion 34 is formed through a selected location of the second surface portion 34 (best shown in FIG. 4). Alternatively, the second surface portion 34 comprises a void which functions as an aperture 36 of the second preferred embodiment (best shown in FIG. 3). In the latter embodiment, a ridge or wall 38 is preferably formed between the first and second surface portions 32, 34 (best shown in FIGS. 3A and 3B). The specific function of this ridge 38 will be explained below.

The aperture 36 serves to provide an opening which allows the Huber needle 14 to be movably accommodated therethrough. As such, the sleeve member 12 is allowed to axially move along the length of the Huber needle 14 toward its distal needle end 16 until the proximal sleeve end 24 approaches the intermediate needle portion 22. When the sleeve member 12 is forced to stop its motion due to the abutment of the proximal sleeve end 24 at the intermediate needle portion 22, the Huber needle 14 along with its distal needle end 16 is displaced from or is released out of the aperture 36.

Upon such displacement from the aperture 36, the distal needle end 16 of the Huber needle 14 becomes contained within the distal sleeve end 26, that is, the cap of the sleeve member 12. Although the distal needle end 16 may be contained within the cap in any manner or fashion, it is preferably shifted away from the aperture 36 and abuts the opposite surface portion from where the aperture 36 is located (as shown in FIGS. 3A and 4A). A slight compression force which exists between the Huber needle 14 and the aperture 36 may operate to cause such lateral or radial shift.

Released from the aperture 36, the sleeve member 12 is prevented from moving back up the Huber needle 14 by abutment with the end wall surface of the cap. Any possibility of inadvertent slippage of the distal needle end 16 into the first embodied aperture 36 is unlikely since the opening of the latter closely conforms to the diameter of the Huber needle 14 (best shown in FIG. 4B). In the instance of the second embodied aperture 36, the presence of the elevated ridge or wall 38 prevents the distal needle end 16 from slipping back into the aperture 36 (best shown in FIG. 3A).

Referring now solely to FIG. 5, an assembly holder device 40 may be optionally used with the safety needle assembly 10 of the present invention. The assembly holder device 40 is utilized for enhancing the securement of the present safety needle assembly 10 to the patient's designated skin area (now shown). In particular, this holder device 40 features an expandible/compressible holder mechanism 42 which is sized and configured to open up and expand in order to accommodate a portion of the sleeve member 12 therewithin.

The holder mechanism 42 may then close to compress the sleeve member 12, and thus be securely engaged thereto. Such closing of the holder mechanism 42 may be achieved in any manner, but preferably is accomplished through folding of two platform wings 43 which individually extend outwardly from the holder mechanism 42 in their unfolded state. Preferably, each of these platform wings 43 are generally rectangular in configuration. The outwardly extending platforms 43 may be taped to the patient's designated skin area so as to firmly mount the safety needle assembly 10 of the present invention thereupon.

In operation, the distal needle end 16 of the Huber needle 14 is first inserted into the patient's designated skin area to access the IV port implanted thereunderneath. As mentioned above, such access is required to perform various tasks such as delivering fluids and medications, drawing blood for diagnostic testing and/or infusing blood products. Optionally, the present needle assembly 10 may be secured in place by engaging the assembly holder device 40 thereto and taping its outwardly extending platforms 43 about the patient's designated skin area.

After IV treatment, the distal needle end 16 of the Huber needle 14 is then withdrawn from the patient. During this procedure, the sleeve member 12 is manually axially pushed toward the distal needle end 16 until the Huber needle 14 becomes displaced from (i.e., released) out of the distal sleeve end's aperture 36. Such axial pushing is preferably performed manually with the non-free hand while the free hand is used to secure the implanted port. The axial travel of the sleeve stops when the proximal end 24 abuts the sharp bend of the intermediate needle section. Similarly during this manually axially moving the sleeve member 12, the distal needle end 16 of the Huber needle 14 is contained within the distal sleeve end 26, thus preventing any risk for a needle-stick injury to its user.

Additional modifications and improvements of the present invention may also be apparent to those of ordinary skill in the art. Thus, the particular combination of parts described and illustrated herein is intended to represent only certain embodiments of the present invention, and is not intended to serve as limitations of alternative devices within the spirit and scope of the invention.

What is claimed is:

1. A safety needle assembly, comprising:
a needle having a distal needle end; and
a sleeve member having a distal sleeve end forming an aperture which accommodates the needle therethrough, the sleeve member being axially movable toward the distal needle end until the needle becomes displaced from the aperture and the distal needle end is contained within the distal sleeve end.

2. The needle assembly of claim 1 wherein the needle is a Huber needle.

3. The needle assembly of claim 1 wherein the distal needle end is non-coring.

4. The needle assembly of claim 1 wherein the sleeve member has a sleeve body which is axially movably engaged around the needle.

5. The needle assembly of claim 4 wherein the sleeve body is fabricated from a flexible material.

6. The needle assembly of claim 1 wherein the sleeve member is manually axially movable along the needle.

7. The needle assembly of claim 1 wherein the needle has a proximal needle portion and a distal needle portion which are interconnected by an intermediate needle portion which disposes the proximal and distal needle portions in a generally perpendicular orientation.

8. The needle assembly of claim 7 wherein the sleeve member has a proximal sleeve end, the sleeve member being axially movable from the proximal needle portion toward the distal needle portion until the proximal sleeve abuts the intermediate needle portion.

9. The needle assembly of claim 8 wherein the proximal sleeve end comprises a collar fabricated from a generally rigid material.

10. The needle assembly of claim 7 wherein the sleeve member has a sleeve length which is sufficient to extend from about the intermediate needle portion to about the distal needle end.

11. The needle assembly of claim 1 wherein the distal sleeve end includes a cap fabricated from a generally rigid material.

12. The needle assembly of claim 11 wherein the cap has an inner cap surface defining first and second surface portions, the distal needle end being disposed toward the first surface portion after becoming displaced from the aperture.

13. The needle assembly of claim 12 wherein the distal needle end abuts the first surface portion when being disposed thereto.

14. The needle assembly of claim 12 wherein a ridge is formed between the first and second surface portions to facilitate the containment of the distal needle end towards the first surface portion.

15. The needle assembly of claim 12 wherein the aperture is formed through the substantial entirety of the second surface portion.

16. The needle assembly of claim 12 wherein the aperture is formed through the second surface portion.

17. The needle assembly of claim 1 further comprising an assembly holder device having an expandible/compressible holder mechanism, the holder mechanism being sized and configured to expand to accommodate a portion of the sleeve member therewithin and be compressed thereafter to be securely engaged thereto.

18. The needle assembly of claim 17 wherein the assembly holder device includes a pair of platforms each extending away from the holder mechanism.

19. A method for containing a needle having proximal and distal needle portions disposed generally perpendicular to each other, the method comprising the steps of:
a) injecting a distal needle end of the needle within the patient;
b) withdrawing the distal needle end from the patient;
c) axially moving a sleeve member having a distal sleeve end with an aperture from the proximal needle portion of the needle toward the distal needle portion thereof;
d) displacing the needle from the aperture of the distal sleeve end; and
e) containing the distal needle end within the distal sleeve end.

20. The method of claim 19 wherein step c) comprises:
1) defining an intermediate needle portion of the needle where the proximal and distal needle portions are interconnected to each other;
2) defining a proximal sleeve end of the sleeve member; and
3) moving the sleeve member manually until the proximal sleeve end abuts the intermediate needle portion.
